(19) United States
(12) Patent Application Publication
(10) Pub. No.: US 2015/0010627 A1
(43) Pub. Date: Jan. 8, 2015

(54) NEW PHARMACEUTICAL COMPOSITIONS OF FLURBIPROFEN AND GLUCOSAMIN

(71) Applicant: Sanovel Ilac Sanayi Ve Ticaret Anonim Sirketi, Istanbul (TR)

(72) Inventors: Umit Cifter, Istanbul (TR); Ali Turkyilmaz, Istanbul (TR); Onur Mutlu, Istanbul (TR); Gaye Ramazanoglu, Istanbul (TR)

(21) Appl. No.: 14/375,716

(22) PCT Filed: Jan. 29, 2013
(86) PCT No.: PCT/TR2013/000046

§ 371 Date: Jul. 30, 2014

(30) Foreign Application Priority Data
Jan. 31, 2012 (TR) 2012-01091
Aug. 17, 2012 (TR) 2012-09601
Jan. 28, 2013 (TR) 2013-01016

(51) Int. Cl. A61K 9/20; A61K 31/7008; A61K 47/36; A61K 47/02; A61K 47/12; A61K 47/38; A61K 31/19; A61K 45/06

(52) U.S. Cl. CPC A61K 9/2086; A61K 31/19; A61K 31/7008; A61K 45/06; A61K 47/02; A61K 47/12; A61K 47/38; A61K 47/36
USPC 424/464; 514/62; 514/53

(57) ABSTRACT

The present invention relates to new pharmaceutical compositions of flurbiprofen or a pharmaceutically acceptable salt thereof and glucosamine or salts thereof. Particularly, the present invention relates to new pharmaceutical compositions for use in the treatment of pain and inflammatory symptoms associated with joint and cartilage disorders, especially with osteoarthritis and rheumatoid arthritis.

### FIELD OF INVENTION

The present invention relates to new pharmaceutical compositions of flurbiprofen or a pharmaceutically acceptable salt thereof and glucosamine or salts thereof. Particularly, the present invention relates to new pharmaceutical compositions for use in the treatment of pain and inflammatory symptoms associated with joint and cartilage disorders, especially with osteoarthritis and rheumatoid arthritis.

### BACKGROUND OF INVENTION

Joint and cartilage disorders, is a painful degenerative condition that results in the deterioration of cartilage tissues that support the weight-bearing joints in the body. Once the cartilage is thinned or lost, the constant grinding of bones against each other causes pain and stiffness around the joint. Abnormal and excess bone formations called spurs grow from the damaged bones, causing further pain and stiffness. It is believed that degenerative joint disorders affect 80% of people over the age of 60. Degenerative joint disorders include, for example, osteoarthritis, rheumatoid arthritis, other rheumatic disorders with cartilage breakdown, chondrolysis after joint trauma, for example, after meniscus or patella injuries or torn ligaments, or chondrolysis associated with prolonged immobilization of joints.

Osteoarthritis is the most prevalent form of arthritis which is a painful, degenerative joint disease that often involves the hips, knees, neck, lower back, or the small joints of the hands. It is characterized by pain and progressive degeneration of cartilage in synovial joints and vertebrae, leading to significant reduction of mobility and quality of life. Osteoarthritis usually develops in joints that are injured by repeated overuse in the performance of a particular job or a favorite sport or from carrying around excess body weight. Eventually this injury or repeated impact thins or wears away the cartilage that cushions the ends of the bones in the joint so that the bones rub together, causing a grating sensation. Joint flexibility is reduced, bony spurs develop, and the joint swells. Usually, the first symptom a person has with osteoarthritis is pain that worsens following exercise or immobility.

Rheumatoid arthritis is an autoimmune inflammatory disease in which the body releases enzymes that attack its own healthy tissues. In rheumatoid arthritis, these enzymes destroy the linings of joints causing pain, swelling, stiffness, deformity, and reduced movement and function. Rheumatoid arthritis also may include systemic symptoms.

Hence, pharmacological treatment of arthritis involves two therapeutic goals:
Analgesic & anti-inflammatory treatment: Relief from pain and inflammation of the soft tissue surrounding the joint.
Disease-modifying treatment to treat the underlying pathology.

Flurbiprofen is a well known, propionic acid derivative, also known as NSAID (non-steroidal anti-inflammatory drug), with the analgesic and anti-inflammatory activities it possesses. It is used in muscle-skeletal and joint disorders such as ankylosing spondylitis, osteoarthritis and rheumatoid arthritis, in soft-tissue disorders such as sprains and strains and for postoperative pains and mild to moderate pain including dysmenorrhoea and migraine. Its chemical structure is illustrated with Formula I given below.

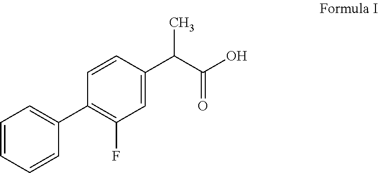 [SMILES: CC(C(=O)O)c1ccc(-c2ccccc2)c(F)c1]

Flurbiprofen is mostly administrated orally in dosages about 150 to 200 mg, may also be increased to 300 mg daily in acute or severe conditions if necessary.

One disadvantage of the oral administration of compositions comprising flurbiprifen, is that the patient is likely to experience unpleasant side effects, including gastrointestinal (GI) adverse effects including inflammation, spontaneous gastric bleeding, ulceration and perforation of the stomach, which can be life threatening. Thus, using flurbiprofen in high dosages may increase the GI adverse effects.

There are various patent applications in prior art in relation to flurbiprofen compositions, for example, U.S. Pat. No. 3,755,427 describes the flurbiprofen molecule and the anti-inflammatory, analgesic, antipyretic and anti-toxic effects of flurbiprofen.

The document U.S. Pat. No. 4,014,993 discloses the use of flurbiprofen in platelet aggregation. The document EP137668 discloses the use of flurbiprofen in the treatment of alveolar bone resorption.

Glucosamine is an amino sugar and aprominent precursor in the biochemical synthesis of glycosylated proteins and lipids. Glucosamine is part of the structure of the polysaccharides chitosan and chitin and it is naturally present in the shells of shellfish, animal bones and bone marrow. It is also present in some fungi and can be also synthetically derived. Glucosamine is used for the treatment of osteoarthritis. Glucosamine may be administered in dosages about 500 to 2500 mg per day.

There are various patent applications in prior art in relation to glucosamine compositions but none of them are specifically used in combination with flurbiprofen in oral administration as tablet or capsule dosage form.

For example, U.S. 2008/0227747 A1 discloses a therapeutic composition and methods for the treatment and prevention of a degenerative joint disorder and/or cardiovascular disease comprising polycosanols, glucosamine and chondroitin. Composition further may comprise NSAIDs, but neither an example nor flurbiprofen as one of the NSAIDs is disclosed in the patent application in combination with glucosamine.

It is well known that drugs used in the same therapeutic area or even for treating the same indication cannot always be combined a priori with the expectation of at least additive therapeutic effects. The scientific literature is full of examples wherein compounds of different classes, which are used to treat the same indications, cannot be combined into safe and efficacious dosage forms thereby resulting in incompatible drug combinations. The reasons for this unexpected lack of compatibility are varied; however, it is often found that the incompatible drug combinations result in increased side effects, unwanted drug interactions or new side effects.

However, not all combinations of NSAIDs and glucosamine are suitable, in terms of safety or efficacy, than the administration of a single product. Thus, no orally-administrable pharmaceutical composition has been produced until today, which contains a combination of flurbiprofen and glucosamine. Even if some medicaments comprising either of these active agents have been administered concomitantly in practice, this fact requires the patients to carry more than one drug and causes application-related difficulties. Additionally, administering and formulating a combination, in place of the individual use of each active agent, may provide improved treatment features.

Another problem is related to combine these two active ingredients in one dosage form such as tablet or capsule, it would require a dosage form having approximately or more than 1000 mg active ingredients in total without any further tablet or capsule excipients. This is an amount that would create a very large tablet or capsule size that would not be swallowable, or it would require composition that would require ingesting multiple tablets to achieve the desired effect.

Accordingly, based on said drawbacks, a novelty is required in the art of pharmaceutical compositions having therapeutic effects against pain and inflammatory symptoms associated with joint and cartilage disorders, especially with osteoarthritis and rheumatoid arthritis.

Therefore, the object of the present invention is to provide new pharmaceutical compositions comprising flurbiprofen and glucosamin for use in the treatment of pain and inflammatory symptoms associated with joint and cartilage disorders, especially with osteoarthritis and rheumatoid arthritis.

### DETAILED DESCRIPTION OF THE INVENTION

The main object of the present invention is to treat, reduce, or prevent the degenerative joint and cartilage disorders by administering to a subject in need thereof a therapeutically effective amount of a composition comprising flurbiprofen and glucosamine, for oral administration, which overcomes the above described problems in prior art and have additional advantages over them.

A further object of the invention is to eliminate the GI adverse effects of flurbiprofen when it is administered orally in high terapeutic effective amounts for a long time. It is known that the treatment of the degenerative joint and cartilage disorders, especially osteoarthritis and rheumatoid arthritis needs a long treatment period. Therefore to use of flurbiprofen for a long time with high terapeutic effective amounts may increase the possibility of GI adverse effects of flurbiprofen. As a rule, after a long-term administration of a drug, drug addiction develops and as a consequence its dosage should be increased. This certainly affects the occurrence of side effects.

The present invention provides the solution to this problem by using not more than 15% flurbiprofen by combining it with glucosamin not less than 45% by weight. It has been found surprisingly that this ratios have an increased/synergistic effect over the flurbiprofen's analgesic and antiinflammatory activity even with low doses.

The role which glucosamine sulfate plays in the treatment or prevention of the degenerative joint and cartilage disorders is most likely associated directly its ability to act as the most important substrate for glycosaminoglycans and a basis of hyaluronic acid. A successful treatment of osteoarthritis and rheumatoid arthritis must control pain effectively as well as slow down or ensure the reverse development of joint degeneration process. It has been found that the introduction of glucosamone sulfate in a quantity of not less than 45% of the total weight of the composition makes it possible to ensure a chondroprotective and anti-inflammatory effect of the composition and prevents destructive effect of glucocorticoids on chondrocytes and to reduce a need for NSAID (i.e. flurbiprofen) in high dosage for patients suffering from osteoarthritis and rheumatoid arthritis which in turn makes it possible to decrease side effect risks.

Accordingly, when flurbiprofen is used for a long period of time, it may have a desensitising effect. It has been also found that when flurbiprofen is used in an amount of not more than 15% by combination with glucosamine not less than 45% of the total weight of the composition makes it possible to ensure increased analgesic and anti-inflammatory effect of the composition, whilst reducing the pain and inflammation syndrome in degenerative joint and cartilage disorders synergisticly. Thus this also reduces the risk of the GI side effects. In one embodiment flurbiprpfen amount is present not more than 10% by weight and glucosamine sulfate amount is present not less than 50% by weight of the total composition.

This ratios also ensure the required effective doses for the therapy without the need of taking the medicine three times a day. In prior art, the formulations comprising glucosamine are taken three times a day. Due to increased tablet weight when trying to increase the required glucosamine effective doses (i.e 750 mg to 1000 mg/tablet or capsule) which should be the minimum 500 mg, occurs some problems during the manufacturing of the composition itself, and for the patient compliance too. Because it would require a dosage form having approximately or more than 1000 mg active ingredients in total without any further tablet or capsule excipients. This is an amount that would create a very large tablet or capsule size that would not be swallowable, or it would require composition that would require ingesting multiple tablets to achieve the desired effect which can be difficult for the patients.

Therefore it has been found that in certain ratios of flurbiprofen to glucosamine which is in the range of 0.010 to 10.0, preferably 0.10 to 5.0, and more preferably 0.10 to 2.0 helps the composition easily processed into a tablet or capsule dosage form, in desired weight which can easily be swallowed by the patients, whilst maintaining or increasing the therapeutic effective doses for the treamnet of joint and cartilage disorders.

According to a preferred embodiment of the present invention, said novelty is realized with the composition comprising, flurbiprofen or pharmaceutically acceptable salts thereof and glucosamine or pharmaceutically acceptable salts thereof.

Flurbiprofen useful in accordance with this invention comprises the pharmaceutically acceptable salts and esters of flurbiprofen, and further includes the conventionally used racemic mixture which comprises the S- and R-enantiomers of flurbiprofen. In a preferred embodiment of the present invention, flurbiprofen is in an amount of 5.0 to 15.0% by weight of the total tablet, preferably it is 5.0 to 10.0% by weight of the total tablet.

The preferred salts of glucosamine in accordance with this invention comprise N-acetyl-glucosamine, glucosamine hydrochloride and glucosamine sulfate and mixtures thereof. In a preferred embodiment of the present invention, the salt is sulfate salt. Glucosamine sulfate . thereof is in an amount of 45.0 to 70.0% by weight of the total tablet, preferably it is 50.0 to 70.0% by weight of the total tablet, more preferably it is 60.0% to 70.0% by weight of the total tablet.

According to a preferred embodiment the pharmaceutical composition of the present invention, therapeutic effective amount of flurbiprofen is present between 100-500 mg/day and therapeutic effective amount of glucosamine sulfate is present between 500-2000 mg/day. In one aspect, flurbiprofen is present in an amount of between 100-300 mg/day and glucosamine sulfate is present in an amount 500-1500 mg/day.

Another preferred embodiment of the present invention comprises at least one or more excipient. According to a preferred embodiment of the present invention, said excipient comprise at least one or more diluents, disintegrants, glidants, lubricants, binders, coloring agents, flavouring agents.

In a preferred embodiment of the present invention, suitable diluents is selected from a group comprising lactose monohydrate, microcrystalline cellulose, corn starch, pregelatinized starch, mannitol, calcium phosphate anhydrate, calcium phosphate dihydrate, calcium phosphate trihydrate, dibasic calcium phosphate, calcium carbonate, calcium sulfate, carboxymethyl cellulose calcium, powdered cellulose, cellulose acetate or mixtures thereof.

In a preferred embodiment of the present invention, suitable disintegrant is selected from a group comprising croscarmellose sodium, hydroxypropyl cellulose, xylitol, crospovidone, low-substituted hydroxypropyl cellulose (L-HPC) and sodium starch glycolate, corn starch or mixtures thereof. In one aspect, disintegrant is present in an amount of from 5.0 to 25.0% by weight of the total tablet composition.

In a preferred embodiment of the present invention, suitable glidant is colloidal silicon dioxide or talc. In one aspect, glidant is present in an amount of from 0.10 to 5.0% by weight of the total tablet composition.

In a preferred embodiment of the present invention, suitable lubricant is selected from the group comprising magnesium stearate, sodium stearyl fumarate, polyethylene glycol, stearic acid, metal stearates, boric acid, sodium chloride benzoate and acetate, sodium or magnesium lauryl sulfate or mixtures thereof. In one aspect, lubricant is present in an amount of from 0.10 to 5.0% by weight of the total tablet composition.

In a preferred embodiment of the present invention, suitable binder is selected from a group comprising polymethacrylate, glyceryl behenate, polyvinylpyrrolidone (povidone), hydroxypropyl methyl cellulose (HPMC), hydroxypropyl cellulose (HPC), carboxymethyl cellulose (CMC), methyl cellulose (MC), hydroxyethyl cellulose, sodium carboxymethyl cellulose (Na CMC), carboxymethyl cellulose calcium, ethyl cellulose and other cellulose derivatives, polyethylene oxide, gelatin, starch, xanthan gum, guar gum, alginate, carrageen, pectin, carbomer, cellulose acetate phthalate, hydroxypropyl starch, hydroxyethyl methyl cellulose, polaxomer, polyethylene glycol (PEG) or mixtures thereof. In one aspect, binder is used optionally and may present in an amount of from 0.10 to 10.0% by weight of the total tablet composition.

In a preferred embodiment of the present invention, suitable coloring agent is selected from a group comprising iron oxides (such as; iron oxide yellow, red or black), Food, Drug & Cosmetic (FD&C) dyes, poncau, indigo blue, indigotine blue, carmoisine indigotine, quinoline yellow, flaming red, carmine, carmoisine, sunset yellow or mixtures thereof. In one aspect, coloring agent is used optionally and may present in an amount of from 0.01 to 1.00% by weight of the total tablet composition.

In a preferred embodiment of the present invention, suitable flavouring agent is selected from a group comprising fruit flavours such as orange, banana, strawberry, cherry, wild cherry, lemon; and other flavours such as cardamom, anise, peppermint, menthol, vanillin and ethyl vanillin or mixtures thereof. In one aspect, flavouring agent is used optionally and may present in an amount of from 0.1 to 2.0 by weight of total composition.

In a preferred embodiment according to the present invention, said pharmaceutical composition ccomprises,
a. flurbiprofen at 5.0-15.0% by weight,
b. glucosmain sulfate at 45.0-70.0% by weight,
c. lactose monohydrate at 10.0-20% by weight,
d. microcrystalline cellulose at 5.0-10% by weight,
e. croscarmellose sodium at 1.0-5.0% by weight,
f. hydroxypropyl cellulose at 1.0-5.0% by weight,
g. colloidal silicon dioxide at 0.10-2.0% by weight,
h. magnesium stearate at 0.10-2.0% by weight,

In another preferred embodiment according to the present invention, said pharmaceutical composition comprises,
a. flurbiprofen at 5.0-10.0% by weight,
b. glucosmain sulfate at 50.0-70.0% by weight,
c. lactose monohydrate at 10.0-20% by weight,
d. microcrystalline cellulose at 5.0-10% by weight,
e. croscarmellose sodium at 1.0-3.0% by weight,
f. hydroxypropyl cellulose at 1.0-3.0% by weight,
g. colloidal silicon dioxide at 0.10-1.0% by weight,
h. magnesium stearate at 0.10-1.0% by weight,

According to another preferred embodiment of the present invention, the flurbiprofen or a pharmaceutically acceptable salts thereof combinations comprising glucosamine is used in the treatment of pain and inflammatory symptoms associated with joint and cartilage disorders, especially with osteoarthritis and rheumatoid arthritis.

According to a further embodiment of the present invention, flurbiprofen and glucosamine sulfate may further be combined with chondroitin sulfate. The therapeutic effective amount of chondroitin sulfate is present between 500-1500 mg/day.

According to this embodiment of the present invention, said pharmaceutical composition comprises,
a. flurbiprofen at 5.0-15.0% by weight,
b. glucosmain sulfate at 45.0-70.0% by weight,
c. chondroitin sulfate at 25-50% by weight,
d. lactose monohydrate at 5.0-20% by weight,
e. microcrystalline cellulose at 1.0-10% by weight,
f. croscarmellose sodium at 1.0-5.0% by weight,
g. hydroxypropyl cellulose at 1.0-5.0% by weight,
h. colloidal silicon dioxide at 0.10-2.0% by weight,
i. magnesium stearate at 0.10-2.0% by weight,

According to a further embodiment of the present invention, flurbiprofen and glucosamine sulfate may further be combined with methylsulfonylmethane or flurbiprofen, glucosamine sulfate and chondroitin sulfate may further be combined with methylsulfonylmethane. The therapeutic effective amount of methylsulfonylmethane is present between 400-1200 mg/day.

According to a further embodiment of the present invention, flurbiprofen and glucosamine sulfate may further be combined with capsaicin or flurbiprofen, glucosamine sulfate and chondroitin sulfate may further be combined with capsaicin.

According to another embodiment of the invention, the pharmaceutical composition is in the form of a tablet or capsule, it may optionally in the form of a bilayer tablet.

According to another preferred embodiment of the present invention, the formulation is orally administered as twice-a-day dosage regimen which increases the patient compliance according to the dosage regimen taken 3 times a day. Thus, patient doesn't need to carry out the tablet or capsule with himself also to prevent the forgotten an omitted dose during the day. Therefore, it is very convenient to take the medicine once in the morning and the second in the evening.

As a further embodiment of the invention, it is possible to prepare tablets or granules by direct compression. Likewise the dry and wet granulation processes are possible as well.

The preferred direct compression process of the present invention for preparing the pharmaceutical composition comprises the following steps;
a. blending flurbiprofen, glucosamine sulphate, microcrystalline cellulose, lactose monohydrate, crosscarmellose sodium and hydroxypropylcellulose progressively, wherein the blending time is preferably 20 min.,
b. adding magnesium stearate and colloidal silicon dioxide to the powder mixture above and blending progressively for about 5 min.
c. compressing the final powder mixture to form tablets, or filling into capsules,
d. optionally coating the tablets.

The preferred dry granulation process of the present invention for preparing the pharmaceutical composition comprises the following steps;
a. mixing flurbiprofen and glucosamine sulfate with lactose monohydrtate, ½ of microcrystalline cellulose, crosscarmellose sodium and hydroxypropylcellulose and granulating the mixture progressively,
b. compacting the blended mixture,
c. adding rest of the microcrystalline cellulose, colloidal silicon dioxide and magnesium stearate to this mixture of step b., and further progressive blending until obtaining a homogenous powder mixture,
d. compressing the blended mixture to form tablets or filling into capsules,
e. optionally coating the tablets.

The preffered wet granulation process for preparing the pharmaceutical composition comprising the following steps;
a. mixing flurbiprofen and glucosamine sulfate with lactose monohydrtate, microcrystalline cellulose, ½ of crosscarmellose sodium and hydroxypropylcellulose and blending,
b. adding water or water+alcohol mixture to this mixture and blending to form granules,
c. sieving and drying the wet granules in oven or fluid bed dryer and sieving the dry granules,
d. rest of the crosscarmellose sodium, colloidal silicon dioxide and magnesium stearate are mixed with the dry granule mixture,
e. compressing the blended mixture to form tablets, or filling into capsules,
f. optionally coating said tablets.

This invention is further defined by reference to the following examples. Although the example is not intended to limit the scope of the present invention, it should be considered in the light of the description detailed above.

### Example 1

### Capsul or Tablet

| Ingredients | % amount |
|---|---|
| flurbiprofen | 8.70 |
| glucosamin sulfate | 65.20 |
| lactose monohydrate | 15.60 |
| microcrystalline cellulose | 7.00 |
| croscarmellose sodium | 1.30 |
| hydroxypropyl cellulose | 1.50 |
| colloidal silicon dioxide | 0.36 |
| magnesium stearate | 0.30 |

The process of the composition is carried out with one of the processes as given above in detail.

### Example 2

### Capsul or Tablet

| Ingredients | % amount |
|---|---|
| flurbiprofen | 8.00 |
| glucosamin sulfate | 45.0 |
| chondroitin sulfate | 30.0 |
| lactose monohydrate | 10.50 |
| microcrystalline cellulose | 3.50 |
| croscarmellose sodium | 1.20 |
| hydroxypropyl cellulose | 1.30 |
| colloidal silicon dioxide | 0.30 |
| magnesium stearate | 0.20 |

The process of the composition is carried out with one of the processes as given above in detail.

1. A pharmaceutical composition for oral administration, comprising flurbiprofen or one or more pharmaceutically acceptable salts thereof and glucosamine or one or more pharmaceutically acceptable salts thereof.

2. The pharmaceutical composition according to claim 1, wherein the one or more pharmaceutically acceptable salts of glucosamine comprises N-acetyl, hydrochloride, sulfate or mixtures thereof, preferably the salt is sulfate salt.

3. The pharmaceutical composition according to claim 1, wherein the amount of flurbiprofen or pharmaceutically acceptable salts thereof is not more than 15% by weight.

4. The pharmaceutical composition according to claim 1, wherein the amount of glucosamine or the one or more pharmaceutically acceptable salts thereof is between 45% and 70% by weight of the total composition.

5. The pharmaceutical composition according to claim 1, wherein the amount of flurbiprofen to glucosamine is in the range of 0.010 to 10.0, preferably 0.10 to 5.0, and more preferably 0.10 to 2.0.

6. The pharmaceutical composition according to claim 1, wherein flurbiprofen is present in an amount of between 100-500 mg/day and glucosamine sulfate is present in an amount of 500-2000 mg/day.

7. The pharmaceutical composition according to claim 1, further comprising at least one or more pharmaceutically acceptable excipients.

8. The pharmaceutical composition according to claim 7 wherein said one or more pharmaceutically acceptable excipients comprise at least one or more diluents, disintegrants, glidants, lubricants, binders, coloring agents, or flavouring agents.

9. The pharmaceutical composition according to claim 8, wherein said one or more dliuents are selected from lactose monohydrate, microcrystalline cellulose, corn starch, pregelatinized starch, mannitol, calcium phosphate anhydrate, calcium phosphate dihydrate, calcium phosphate trihydrate, dibasic calcium phosphate, calcium carbonate, calcium sulfate, carboxymethyl cellulose calcium, powdered cellulose, cellulose acetate and mixtures thereof

10. The pharmaceutical composition according to claim 8, wherein said one or more disintegrants are selected from croscarmellose sodium, hydroxypropyl cellulose, xylitol, crospovidone, low-substituted hydroxypropyl cellulose (L-HPC), sodium starch glycolate, corn starch and mixtures thereof

11. The pharmaceutical composition according to claim 8, wherein said one or more glidants is colloidal silicon dioxide or talc.

12. The pharmaceutical composition according to claim 8, wherein said one or more lubricants are selected from magnesium stearate, sodium stearyl fumarate, polyethylene glycol, stearic acid, metal stearates, boric acid, sodium chloride benzoate and acetate, sodium or magnesium lauryl sulfate and mixtures thereof.

13. The pharmaceutical composition according to claim 1, comprising,
a. flurbiprofen at 5.0-15.0% by weight,
b. glucosmain sulfate at 45.0-70.0% by weight,
c. lactose monohydrate at 10.0-20% by weight,
d. microcrystalline cellulose at 5.0-10% by weight,
e. croscarmellose sodium at 1.0-5.0% by weight,
f. hydroxypropyl cellulose at 1.0-5.0% by weight,
g. colloidal silicon dioxide at 0.10-2.0% by weight, and
h. magnesium stearate at 0.10-2.0% by weight.

14. The pharmaceutical composition according to claim 1, further comprising chondroitin sulfate.

15. The pharmaceutical composition according to claim 14, comprising,
a. flurbiprofen at 5.0-15.0% by weight,
b. glucosmain sulfate at 45.0-70.0% by weight,
c. chondroitin sulfate at 25-50% by weight,
d. lactose monohydrate at 5.0-20% by weight,
e. microcrystalline cellulose at 1.0-10% by weight,
f. croscarmellose sodium at 1.0-5.0% by weight,
g. hydroxypropyl cellulose at 1.0-5.0% by weight,
h. colloidal silicon dioxide at 0.10-2.0% by weight, and
i. magnesium stearate at 0.10-2.0% by weight.

16. The pharmaceutical composition according to claim 1, further comprising methylsulfonylmethane.

17. The pharmaceutical composition according to claim 1, further comprising capsaicin.

18. The pharmaceutical composition according to claim 1, wherein the composition is in the form of a tablet or capsule.

19. The pharmaceutical composition according to claim 18, wherein the composition is in the form of a bilayer tablet.

20. (canceled)

21. A method for the treatment of pain and inflammatory symptoms associated with joint and cartilage disorders in a patient, comprising administering an effective amount of the pharmaceutical composition of claim 1 to the patient in need thereof.
